(19) United States
(12) Patent Application Publication
(10) Pub. No.: US 2021/0046399 A1
(43) Pub. Date: Feb. 18, 2021

(54) Near-Shore Floating Methanol Conversion Ship and Export Terminal

(71) Applicant: NW Innovation Works., Kalama, WA (US)

(72) Inventors: Simon Zhang, Kalama, WA (US); Hongjun Henry Ji, Rancho Santa Fe, CA (US); Yanning Luo, Shanghai (CN)

(21) Appl. No.: 16/964,963

(22) PCT Filed: Jan. 24, 2019
(86) PCT No.: PCT/US2019/014951

§ 371 Date: Jul. 24, 2020

(51) Int. Cl. B01D 3/14; B01D 1/14; B01J 8/02

(52) U.S. Cl. CPC B01D 3/14; B01D 1/14; B63B 2035/4473; B01J 2219/00018; B01J 8/02

(57) ABSTRACT

There is disclosed a near shore floating vessel for large scale production of methanol (capable of producing at least 4000 tons per 24-hour day) from natural gas (methane) and for export shipment. More specifically, the near shore floating vessel obtains methane from an on-shore methane stream or pipeline. The disclosed near-shore floating vessel provides several environmental and commercial advantages to move methane export to a near shore instead of an on-shore location.

### CROSS REFERENCE TO RELATED APPLICATION

This patent application claims priority to U.S. Provisional Patent Application 62/621,318 filed 24 Jan. 2018.

### TECHNICAL FIELD

The present disclosure provides a near shore floating vessel for large scale production of methanol (capable of producing at least 2000 tons per 24-hour day) from natural gas (methane). More specifically, the near shore floating vessel obtains methane from an on-shore methane stream or pipeline. More specifically, the present disclosure provides several environmental and commercial advantages to move methane export to a near shore instead of an on-shore location.

### BACKGROUND

Methanol production from methane sources has been done on smaller scales as the process begins to satisfy economic and environmental considerations. North America has a surplus of methane, much of which is stranded and is flared off by burning it to generate CO₂ as methane released into the atmosphere is a more potent greenhouse gas that can impact climate change (irrespective of what the current US federal administration claims). But flaring off natural gas is a loss of income for oil and gas producers that could be recovered by selling the natural gas/methane product. But natural gas demand in North America is not sufficient in view of increasing supply, so the laws of economics indicate the price goes down. This economic and environmental problem is looking for a solution by means of increasing exports from North America of surplus methane.

Methanol is a major chemical raw material. Present global consumption is about 27 million tons per year. Major uses of methanol include the production of acetic acid, formaldehyde, and methy-t-butylether. The latter, an oxygenate additive to gasoline, has accounted for about a third of all use. Worldwide demand for methanol is expected to increase as much as five-fold over the next decade as potential new applications become commercialized. Such applications include, for example, the conversion of methanol to light olefins, the use of methanol for power generation, and the use of methanol for fuel-cell powered automobiles.

Natural gas is a hydrocarbon gas in private homes as well as in the industry, and is also used for electric power generation, as well as being used as fuel for transportation purposes. Natural gas is often piped from the gas production source to the consumers, but over longer distances transporting the natural gas at sea proves economically favorable. The ability to transport the gas also enables provision of gas to distant markets. However, it is often prohibitively expensive to provide liquified natural gas (LNG) terminals in order to compress methane into a liquid form.

Other solutions have tried creating FLNGtis (floating liquified natural gas vessels), that are also prohibitively expensive, but can float off-shore. The compressed liquified natural gas made in a FLNGV is designed to either also deliver the liquified natural gas cargo or conduct a ship-to-ship transfer. However, there is a need for a wide distance or safety zone, in the case of an LNG (liquified natural gas) spill which could result in severe structural damage, including steel structures and immediate personnel fatalities. Therefore, there is a need to improve the process for international shipments of natural gas, such as to make methanol instead of LNG and to move the process off-shore so as to avoid shoreline management issues, and issues associated with limited industrial deep-water port access.

U.S. Pat. No. 4,134,732 provides for a low capacity floating plant to produce methanol from offshore natural gas wells. This floating plant can only be operated during calm seas (good weather) because of tall distillation columns for methanol purification. This small scale floating process stores liquid methanol between a methanol synthesis reactor and a tall distillation column such that it cannot provide for larger scale export production of methanol from onshore natural gas supply. This system is designed to be mobile and transported to offshore wells to capture methane rather than flare it off. There is a need in the art to design floating methanol production from onshore natural gas as a means for exporting excess natural gas instead of processing methane off wells instead of flaring it.

Methanol synthesis is based on the equilibrium reactions of syngas. Syngas is defined as a gas comprising primarily carbon monoxide (CO), carbon dioxide (CO₂) and hydrogen (H₂). Other gases present in syngas include methane (CH₄), and small amounts of light paraffins, such as ethane and propane. One way of characterizing the composition of a syngas stream for methanol synthesis is to account for the CO₂ present in the syngas stream.

The initial step in the production of methanol is to produce syngas from a methane-containing gas, such as natural gas or refinery off-gas. The associated costs of producing the syngas accounts for over half of the capital investment in the methanol plant. The syngas can be generated using steam methane reforming or partial oxidation reforming which includes combined reforming or autothermal reforming.

In UK Patent Application GB 2092172A partial oxidation reformers are used in the production of syngas for the production of synthetic hydrocarbons, that is, Fischer-Tropsch type conversion, often produces an excess quantity of CO₂ that eventually must be removed from the process stream. Consequently, there are associated costs in producing and removing the CO₂. Excess CO₂ produced by the partial oxidation reformer can be utilized in part by first passing the syngas to a methanol synthesis reactor prior to the hydrocarbon synthesis reactor. The methanol synthesis utilizes the CO₂ as a carbon source to produce methanol. Alternatively, the CO₂ can be mixed with hydrogen, produced from an external source, to convert the CO₂ to more CO according to the water-gas shift reaction. The additional CO₂ is then used to produce more synthetic hydrocarbon.

U.S. Pat. No. 5,177,114 teaches the conversion of natural gas to methanol or methanol and synthetic hydrocarbons using a relatively low-cost, self-sufficient process. The natural gas is mixed with a 1:1 O₂/N₂ stream at elevated temperatures and pressures to produce a reform gas, which is then used to produce methanol and/or synthetic hydrocarbons. The natural gas is converted without the need for a costly steam reformer or a partial oxidation reformer. Also, the process is directed to low carbon conversions, e.g., about 50 to 65%, so that the tail gas from the process can be used to drive the compressors and other energy intensive units in the process.

A commercial process for the production of methanol starts with the generation of synthesis gas containing carbon monoxide and hydrogen. When natural gas is the raw material, synthesis gas can be formed by reacting the methane in the natural gas with carbon dioxide and water over a catalyst at elevated temperatures. The resulting synthesis gas is converted to methanol at high pressures using a suitable catalyst.

Therefore, there is a need in the art to be able to produce methanol from natural gas without causing any shoreline or environmental disruption. The present disclosure was made to address the foregoing economic energy and chemical resource balancing and environmental concerns by moving methanol production from on-shoreline to a near-shore floating production and export system.

### SUMMARY

1. The present disclosure provides a near shore floating methanol conversion system to produce methanol from natural gas (methane) on its deck with storage of liquid methanol for export within its tanker hold. Preferably, a near-shore location means an average tidal depth of from about 10 meters to about 100 meters average water depth and having a mooring system connected to an on-shore natural gas pipeline. More preferably, the near shore location is an average water depth of 15 meters to 60 meters. Preferably, the on-deck methanol system comprises inputs of natural gas, steam and oxygen and has GHR (catalyst is loaded in the tube) (1), an ATR (2), a Heat recovery system (3), an Air separation Unit (ASU) (4), a Boiler (5), a Syngas compressor unit (6), a Methanol synthesis unit (7), and a Methanol distillation system (8) all connected as shown in FIG. 1.

The present disclosure provides a near shore floating vessel for large scale production of methanol (capable of producing at least 4000 tons per 24-hour day) from natural gas (methane) and for export shipment. More specifically, the near shore floating vessel obtains methane from an on-shore methane stream or pipeline. More specifically, the present disclosure provides several environmental and commercial advantages to move methane export to a near shore instead of an on-shore location. Moreover, the present near shore floating vessel is capable of producing about 5000 tons per day of methanol with access to one natural gas pipeline, in contrast to being able to service sallow gas produced at off-shore wells. The shallow gas can be piped on-shore and gathered into natural gas systems.

The near shore floating methanol conversion system comprising a deck and a hull having a hold, wherein the deck comprises:

(a) an access port to a natural gas line to supply natural gas to the floating near shore methanol conversion system;

(b) a reformer system to convert natural gas to a syngas;

(c) a methanol converter and distillation system to form methanol by oxidation of the syngas; and

(d) a holding tank within a hull of the floating methanol conversion system.

Preferably, the near shore floating methanol conversion system further comprises a mooring system having a natural gas termination line from an on-shore location. More preferably, the mooring further comprises access to electrical power.

Preferably, the reformer system comprises a steam reformer connected to an ATR (autothermal reformer) to produce syngas from an input of natural gas and steam to the steam reformer and oxygen to the ATR. More preferably, the oxygen input to the ATR comprises an ASU (air separation unit) to generate pure oxygen and avoid nitrogen input into the reformer system.

### BRIEF DESCRIPTION OF THE FIGURES

FIG. 1 shows a schematic of an on-deck methanol production system and their connections of the components, comprising inputs of natural gas, steam and oxygen and has GHR (catalyst is loaded in the tube) (1), an ATR (2), a Heat recovery system (3), an Air separation Unit (ASU) (4), a Boiler (5), a Syngas compressor unit (6), a Methanol synthesis unit (7), and a Methanol distillation system (8) all connected as shown in FIG. 1.

FIG. 2 shows a diagram of a preferred methanol from methane/natural gas conversion system to can produce 5000 tons per day of methanol on a ship substantially the size of a New Panamax or Suezmax tanker.

FIG. 3 is a labelled depiction of a floating methanol conversion ship moored to a yoke with a natural gas line that shows the configuration of the FIGS. 1 and 2 components on the deck of the ship.

### DETAILED DESCRIPTION

### Methanol Production Process

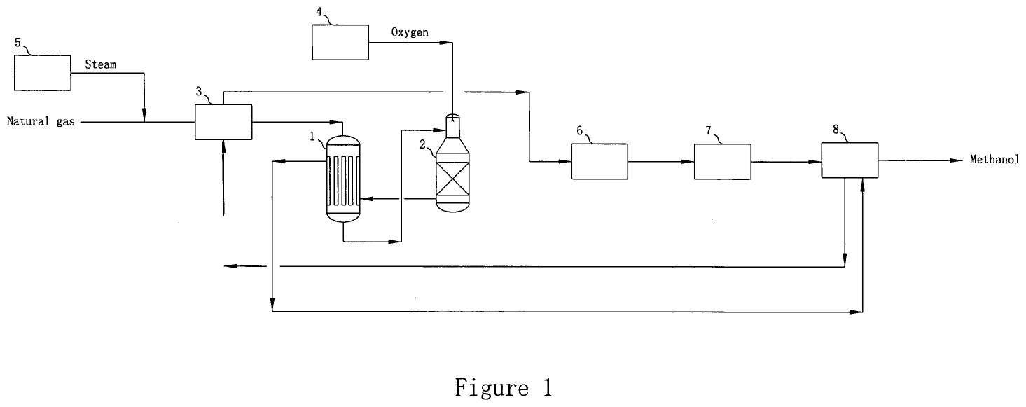

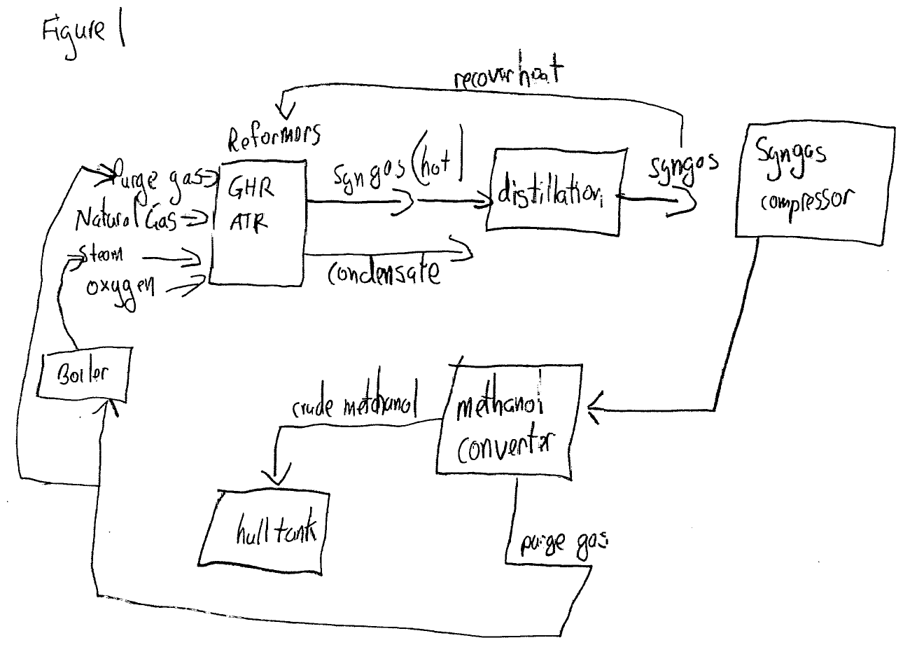

A floating methanol conversion system preferably uses a dual reformer system, such as that shown in FIG. 1 schematically and in FIG. 2 in a pictorial presentation. The dual reformer uses a steam reformer (labeled “GHR” for gas heated reforming) with heat exchangers to transmit heat to an ATR (autothermal reformer). The inputs to the dual reformer (FIGS. 1 and 2) is natural gas (methane with —SH added so that there will be an odor to detect it), steam produced by a boiler and oxygen to the ATR component of the dual reformer system. The oxygen gas is preferably substantially pure oxygen or an oxygen-enriched air. The amount of oxygen use is preferably provides an oxygen:carbon molar ratio is the range of 0.5 to 0.75 to 1. Steam is at a carbon ratio between 0.6 and 2. The syngas produced is hot and the heat is recovered to utilize back in the reformer to reduce heat needed to run the reforming reaction and minimize CO₂ emissions from burning natural gas to produce heat. Moreover, the key to this example of a dual reformer system is that it allows for greater output capacity while minimizing surface area foot print on a limited deck surface for a floating methanol system.

Steam is provided directly to the combustion zone of the steam reformer. The methane in the feed gas is partially oxidized by oxygen from the oxygen enriched stream in the ATR or autothermal reformer. The partial oxidation reactions (with substantially pure O₂) are exothermic so the partial oxidation reactions raise the temperature of the reformed gas to 1200 to 1500° C.

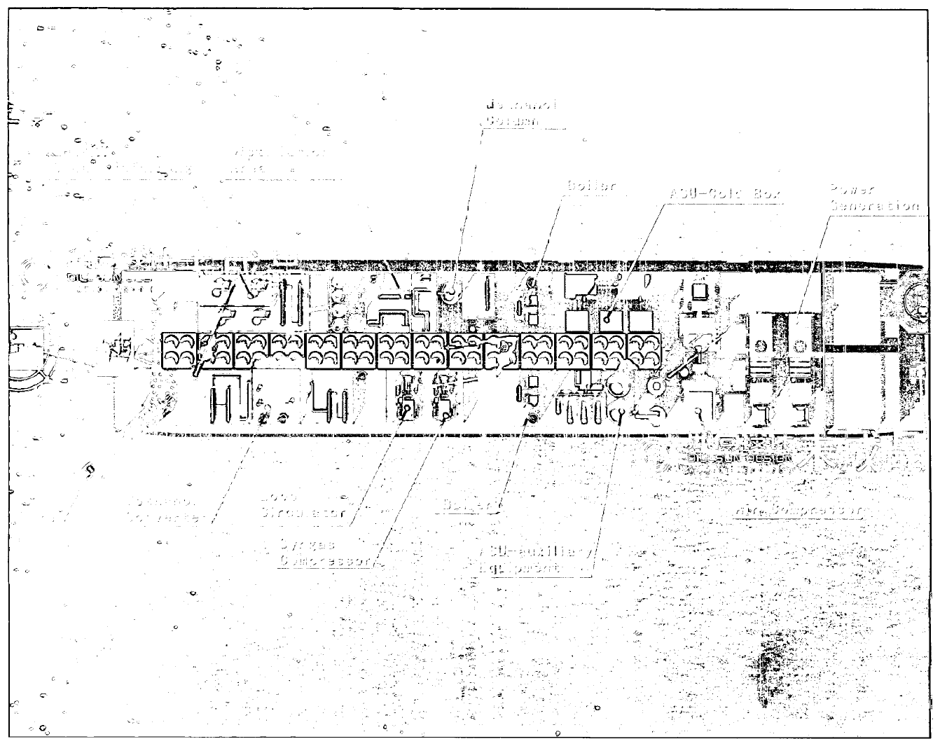

This hot syngas from the ATR reformer is moved a short distance on the deck of the floating ship (FIG. 3) to both a distillation column and a syngas compressor to move both syngas and heat to be utilized without requiring additional heat sources from burning natural gas (and creating CO₂ emissions). This process is in contrast to a much lower capacity U.S. Pat. No. 7,799,834 which uses tall distillation columns that limit the capacity of its floating system and uses air (with majority inert nitrogen) to significantly limit the capacity of the floating vessel to produce methanol to converting gas from well heads to prevent flaring rather than providing a means for exporting natural gas (that competes with LNG (liquified natural gas) export systems).

FIG. 3 is a picture of an exemplary floating methanol production ship for commercial quantity methanol production of from about 5000 tons per day to a larger version of about 10,000 tons per day. The components shown in FIG. 1 in a diagram are placed on the deck of the disclosed methanol converter ship. The hold tank is not shown but located underneath the deck to hold liquid methanol output that is either moved directly with the disclosed ship or off-loaded to a tanker ship in a sea-to-sea transfer of the liquid methanol. The ASU cold box produces pure oxygen to feed into the reformers. The boiler produces steam to feed into the reformers. The overall size of the ship shown in FIG. 3 is substantially similar to a Panamax tanker that can fit through the Panama Canal and is capable of producing about 5000 tons of methanol per day (24 hours) with one natural gas line feeding the ship via a mooring shown in FIG. 3. FIG. 3 also shows an optional power generator to allow for methanol production without power being supplied through the mooring. Therefore, the disclosed methanol ship can produce export quantities (about 5000 to about 10,000 tons per day of methanol with one natural gas lines or two natural gas lines) as a floating near shore production ship for export quantities of methanol.

1-4. (canceled)

5. A near shore floating methanol conversion system comprising a deck and a hull having a hold, wherein the deck comprises:
(a) an access port to a natural gas line to supply natural gas to the floating near shore methanol conversion system;
(b) a reformer system to convert natural gas to a syngas;
(c) a methanol converter and distillation system to form methanol by oxidation of the syngas; and
a holding tank within a hull of the floating methanol conversion system.

6. The near shore floating methanol conversion system of claim 5, further comprising a mooring system having a natural gas termination line from an on-shore location.

7. The near shore floating methanol conversion system of claim 6, wherein the mooring further comprises access to electrical power.

8. The near shore floating methanol conversion system of claim 5, wherein the reformer system comprises a steam reformer (SMR) or a SMR connected to an ATR (autothermal reformer) to produce syngas from input of natural gas and steam to the steam reformer and oxygen to the ATR.

9. The near shore floating methanol conversion system of claim 8, wherein the oxygen input to the ATR comprises an ASU (air separation unit) air to generate pure oxygen and avoid nitrogen input into the reformer system.
